(19) United States
(12) Patent Application Publication
(10) Pub. No.: US 2005/0152972 A1
(43) Pub. Date: Jul. 14, 2005

(54) Soft chewable anesthetic lozenges

(76) Inventor: Mohinder Singh, Naperville, IL (US)

Correspondence Address: LADAS & PARRY LLP, 224 SOUTH MICHIGAN AVENUE, SUITE 1200, CHICAGO, IL, 60604, US

(21) Appl. No.: 10/757,067

(22) Filed: Jan. 14, 2004

(51) Int. Cl. A61K 9/20; A61K 31/24; A61K 31/045

(52) U.S. Cl. 424/464; 514/537; 514/729

(57) ABSTRACT

A soft, chewable lozenge for relief of mouth pain contains, by weight, 10-60% hydrogentated starch hydrolysate, 3-60% hydrogenated mono- and di-saccharides, 1-20% hydrogenated vegetable oil, 0.5-27% gelatin, 0.25-7.5% anesthetic and 1-25% water. A method for treating mouth and throat pain by administering to a patient in need a soft, chewable, non-sticky anesthetic lozenge is disclosed.

### BACKGROUND OF THE INVENTION

The invention relates to the field of topical anesthetic compositions and method for treatment of mouth pain.

Numerous topical anesthetics compositions are known in the art for treatment of mouth and throat pain. Traditionally, such compositions have been based on hard candies which are satisfactory for treatment of throat pain, but which tend to irritate mouth sores when used for treatment of mouth pain.

Other types of mouth treatments are also known, including those based on chewing gum, as disclosed in U.S. Pat. Nos. 4,853,212, 4,983,405 and 4,822,597. Chewable gelatin capsules are also known, as disclosed in U.S. Pat. No. 4,428,927, these capsules also containing a masticatory material such as chicle.

### SUMMARY OF THE INVENTION

It is an object of the invention to provide an oral anesthetic based on a soft chewable material which disintegrates and dissolves slowly in the mouth to sooth the irritated area, and which is non-irritating to sensitive areas of the mouth.

It is a further object of the invention to provide an oral anesthetic composition which contains anesthetic distributed throughout the composition.

It is a still further object of the invention to provide an oral anesthetic composition which does not contain sugar in order to benefit the diabetic population.

It is another object of the present invention to provide a method of treating mouth and throat pain with a soft, chewable, non-sticky anesthetic lozenge.

To achieve these and other objects the invention is directed to a soft, chewable oral anesthetic composition comprising, by weight:

| | |
|---|---|
| hydrogenated starch hydrolysate | 10-60% |
| hydrogenated mono- and di-saccharides | 3-60% |
| hydrogenated vegetable oil | 1-20% |
| gelatin | 0.5-27% |
| anesthetic | 0.25-7.5% |
| water | 1-25%. |

### DETAILED DESCRIPTION OF THE INVENTION

The form of the present product is based upon a combination of hydrogenated starch hydrolysate, hydrogenated mono- and di-saccharides, gelatin and water.

The hydrogenated starch hydrolysate and the hydrogenated mono- and di-saccharides are both starch-based materials solid which dissolve slowly in the mouth, and which provide sweetness to the product. The gelatin, which is added as a water solution, makes the product soft and flexible to prevent irritation of mouth sores. The hydrogenated starch hydrolysate is added in an amount of 10-60% by weight, preferably 40-45%, and the hydrogenated mono- and di-saccharides are added in an amount of 3-60% by weight, preferably 12-20%. The gelatin is added in an amount of 0.5-27% by weight, preferably 8-12%.

The preferred anesthetics for the compositions of the invention are benzocaine in an amount of 0.25 to 7.5% by weight, preferably 0.5-2.5%, and menthol in an amount of 0.25 to 3% by weight, preferably 0.2-0.5%. Mixtures of anesthetics are acceptable, and any other conventionally used oral anesthetic may be used providing it is compatible with these compositions. Preferably, a combination of benzocaine and menthol will be used, the menthol providing both external analgesic action and a pleasant taste.

The polyethylene glycol is added to solubilize the benzocaine, which is not water-soluble. Polyethylene glycol is also preferably present in an amount of 0.05 to 5% by weight, preferably 0.3-0.6%, the polyethylene glycol having a chain length of between 4 and 800, depending on the properties to be imparted to the product. PEG-75 is preferred.

While the hydrogenated starch hydrolysate is a polyhydric alcohol (carbohydrate) which may provide some sweetening, it is preferred to add a further non-sugar sweetener such as sucralose in an amount of 0.01-1.5% by weight, preferably 0.2-0.5%.

The compositions also preferably include flavoring in an amount of 0.1 to 7% by weight, preferably 1-2.5%, and silica in an amount of 0.5 to 10% by weight, preferably 1.5-3.0%. The silica, which is added to reduce stickiness and prevent rapid dissolution of the product, may be amorphous silica, fumed silica, and other forms of silica suitable for use in edible compositions.

Hydrogenated lecithin is added as an emulsifier and mold releaser, and is preferably present in an amount of 0.1 to 5% by weight, preferably 0.1-0.3%. Hydrogenated lecithin acts as a co-emulsifier between the hydrophilic and lipophilic phases of the composition.

Hydrogenated vegetable oil, preferably hydrogenated coconut oil, is added to lubricate the mold used to form the product, to facilitate release from the mold, and to reduce the stickiness of the composition. It is added in an amount of 1-20% by weight, preferably 7-10%.

Preservatives will also be present, for example tetrasodium EDTA, tetrahexyldecyl ascorbate and citric acid (also a buffer and flavoring), the amount of preservative being in the range of 0.04 to 5% by weight, preferably 0.05-0.5%.

The lozenges of the invention are prepared by mixing the gelatin with water heated to 55-60° C., with continuous sweep and air mixing until no lumps are present, forming a gelatin phase. In a separate container, the hydrogenated starch hydrolysate and hydrogenated mono- and di-saccharides are mixed with water and heated until the mixture reaches 75-80° C. and all solids are dissolved. This mixture is added to the gelatin solution with continuous mixing at 60-65° C., and the hydrogenated vegetable oil is added, followed sequentially by the silica and hydrogenated lecithin.

Separately, the polyethylene glycol and benzocaine are heated to 60-65° C. until the solution is clear, and this is added to the gelatin phase with continuous mixing.

Separately, the menthol, flavor and sucralose are mixed until a smooth suspension is obtained, and this suspension is added to the gelatin phase.

Finally, citric acid is added to the gelatin phase, and mixing is continued until a clear product is obtained, which is filled into tray molds which are sealed with a lid using heat and pressure, and then cooled by passing through a cold chamber.

In order to keep this product fresh and soft, the lozenges should be individually wrapped, preferably in clear plastic with strong barrier properties, or a combination of plastic with a peelable foil.

### EXAMPLE

An anesthetic lozenge is formulated containing, in weight %:

| | |
|---|---|
| Hydrogenated starch hydrolysate | 48.530 |
| (Lycasin) | |
| Purified water | 14.100 |
| Hydrogenated mono- and di-saccharides | 13.000 |
| (Isomalt STM: 6-0-δ-D-glucopyranosyl-D- | |
| sorbitol (1,6-GPS) and 1-0-δ-D- | |
| glucopyranosyl-D-mannitol dihydrate | |
| (1,1-GPM dihydrate) | |
| Gelatin 250 BLOOM | 10.000 |
| Hydrogenated coconut oil | 8.145 |
| Silica | 2.500 |
| Apple flavor | 1.500 |
| Benzocaine | 0.750 |
| PEG-75 | 0.450 |
| Menthol USP | 0.375 |
| Sucralose (1,6-dichloro-1,6-deoxy-β-D- | 0.350 |
| fructofuranosyl-4-chloro-4-deoxy-α-D- | |
| galactopyranoside) | |
| Hydrogenated lecithin (Lucinol S-10) | 0.200 |
| Citric acid | 0.100 |
| Total: | 100.000 |

The foregoing ingredients were prepared to yield a soft, chewable, non-sticky, sugar-free anesthetic lozenge.

The inventive formulations are useful in treating mouth and throat pain. A method of treating mouth and throat pain is accomplished by administering to the mouth and throat of a patient in need thereof, a soft, chewable, non-sticky anesthetic lozenge comprising by weight: hydrogentated starch hydrolysate 10 to 60%, hydrogenated mono- and di-saccharides 3 to 60%, hydrogenated vegetable oil 1 to 20%, gelatin 0.5 to 27%, anesthetic 0.25 to 7.5%, water 1 to 25%. The anesthetic composition may include benzocaine in amounts ranging from 0.25% to 7.5% by weight, menthol in an amount of 0.25 to 3% by weight and mixtures. The composition may contain a sweetner, preferably sucralose. The composition may also optionally contain the following ingredients: silica in an amount of 0.5 to 10% by weight, flavoring in an amount of 0.1 to 7% by weight and hydrogenated vegetable oil in an amount of 1 to 20% by weight.

While there have been described what are presently believed to be the preferred embodiments of the present invention, those skilled in the art will realize that changes and modifications may be made thereto without departing from the spirit of the invention, and it is intended to claim all such changes and modifications as that fall within the true scope of the invention.

1. A soft, chewable anesthetic lozenge, comprising, by weight:

| | |
|---|---|
| hydrogenated starch hydrolysate | 10-60% |
| hydrogenated mono- and di-saccharides | 3-60% |
| hydrogenated vegetable oil | 1-20% |
| gelatin | 0.5-27% |
| anesthetic | 0.25-7.5% |
| water | 1-25%. |

2. The lozenge of claim 1, wherein the anesthetic is selected from the group consisting of benzocaine in an amount of 0.25 to 7.5% by weight, menthol in an amount of 0.25 to 3% by weight, and mixtures thereof.

3. The lozenge of claim 1, additionally comprising a sweetener.

4. The lozenge of claim 3, wherein the sweetener is sucralose.

5. The lozenge of claim 1, additionally comprising silica in an amount of 0.5 to 10% by weight.

6. The lozenge of claim 1, additionally comprising 0.1 to 7% by weight of flavoring.

7. The lozenge of claim 1, wherein the hydrogenated vegetable oil is present in an amount of 1-10% by weight.

8. The lozenge of claim 1, additionally comprising 0.5 to 5% by weight of polyethylene glycol of between PEG-4 and PEG-800.

9. The lozenge of claim 8, wherein the polyethylene glycol is PEG-75.

10. A method of treating mouth and throat pain comprising administering to the mouth and throat a soft, chewable, non-sticky anesthetic lozenge comprising, by weight: hydrogentated starch hydrolysate 10 to 60%, hydrogenated mono- and di-saccharides 3 to 60%, hydrogenated vegetable oil 1 to 20%, gelatin 0.5 to 27%, anesthetic 0.25 to 7.5%, water 1 to 25%.

11. The method of claim 10 wherein the anesthetic is selected from the group consisting of benzocaine in an amount of 0.25 to 7.5% by weight, menthol in an amount of 0.25 to 3% by weight, and mixtures thereof.

12. The method of claim 10 wherein the lozenge additionally comprises a sweetner.

13. The method of claim 11 wherein the sweetner is sucralose.

14. The method of claim 10 wherein the lozenge additionally comprises silica in an amount of 0.5 to 10% by weight.

15. The method of claim 10 wherein the lozenge additionally comprises 0.1 to 7% by weight of flavoring.

16. The method of claim 10 wherein the lozenge additionally comprises 1 to 20% by weight of hydrogenated vegetable oil.
